(19) United States
(12) Patent Application Publication
(10) Pub. No.: US 2026/0014107 A1
(43) Pub. Date: Jan. 15, 2026

(54) FEED FOR ANADROMOUS FISH, METHOD FOR PRODUCING IT, METHOD OF INCREASING SEAWATER TOLERANCE, GROWTH AND FEED INTAKE OF ANADROMOUS FISH, FISH FEED FOR USE IN PREVENTING OR REDUCING SEVERITY OF CATARACTS

(71) Applicant: Nutreco IP Assets B.V., Boxmeer (NL)

(72) Inventors: Vibeke VIKESÅ, STAVANGER (NO); Saravanan SUBRAMANIAN, STAVANGER (NO); May-Helen HOLME, STAVANGER (NO); Gunvor STRUKSNÆS, STAVANGER (NO)

(73) Assignee: Nutreco IP Assets B.V., Boxmeer (NL)

(21) Appl. No.: 18/880,037

(22) PCT Filed: Jun. 30, 2023
(86) PCT No.: PCT/NO2023/060008

§ 371 Date: Dec. 30, 2024

(30) Foreign Application Priority Data
Jul. 1, 2022 (NO) 20220763

(51) Int. Cl. A61K 31/198; A23K 20/142; A23K 20/158; A23K 20/22; A23K 20/24; A23K 40/25; A23K 50/80; A61K 9/20; A61K 31/4172; A61K 33/14; A61P 3/00; A61P 27/12

(52) U.S. Cl. CPC A61K 31/198; A23K 20/142; A23K 20/158; A23K 20/22; A23K 20/24; A23K 40/25; A23K 50/80; A61K 9/2013; A61K 9/2063; A61K 9/2068; A61K 9/2095; A61K 31/4172; A61K 33/14; A61P 3/00; A61P 27/12

(57) ABSTRACT

A fish feed for feeding to anadromous fish in freshwater having arginine in free amino acid form or dipeptide form, 0.1 to 7 wt % histidine in free amino acid form or dipeptide form, and 0.2-5 wt % Na+. The fish feed may be fed to anadromous fish to promote growth in the freshwater and/or seawater phase, to promote feed intake and/or to prevent or reduce severity of cataracts.

### CROSS-REFERENCE TO RELATED APPLICATIONS

This application is the U.S. national stage application of International Application No. PCT/NO2023/060008, filed Jun. 30, 2023, which international application was published on Jan. 4, 2024, as WO 2024/005653 in the English language. The International Application claims priority to Norwegian patent application No. 20220763, filed Jul. 1, 2022. The international application and Norwegian application are both incorporated herein by reference, in their entirety.

### FIELD

The invention relates to a feed for anadromous fish, methods of using the feed, and a method of making the feed.

### BACKGROUND

Fish is an important source of protein for the world's population. It is recognised that consumption of fish per capita should be increased because of its positive health effects.

However, it is no longer possible to increase the quantity of fish caught in the wild, because of the effect on fish stocks. Some stocks of wild fish have collapsed already, and for other stocks the catch must be reduced for the stocks to be sustainable.

Aquaculture (fish farming) is therefore of increasing importance in supplying fish to the world's population.

Fish need protein, fat, minerals and vitamins in order to grow and to be in good health. The diet of carnivorous fish, such as salmonids, is particularly important.

Originally in the farming of carnivorous fish, whole fish or ground fish were used to meet the nutritional requirements of the farmed fish. Ground fish mixed with dry raw materials of various kinds, such as fish meal and starch, was termed soft or semi-moist feed. As farming became industrialized, soft or semi-moist feed was replaced by pressed dry feed. This was itself gradually replaced by extruded dry feed.

Today, extruded feed is nearly universal in the farming of salmonids.

The dominant protein source in dry feed for fish has been fish meal of different qualities. Fish meal and fish oil are obtained from so-called “industrial fish”. The catch of industrial fish cannot be increased. Industrial fish may for example be of North-European origin or of South-American origin, in particular fish caught off the coasts of Peru and Chile. The output of these countries fluctuates somewhat from one year to the next. At about 7 year intervals the weather phenomenon El Niño occurs, and severely reduces the output of industrial fish. This affects the availability of fish meal and fish oil on the world market, and prices rise considerably for these raw materials.

The aquaculture industry and especially the fish feed industry have predicted for some years that there will be a shortage relative to demand of both fish meal and fish oil in the future.

Other animal protein sources are also used for dry fish feed. Thus, it is known to use blood meal, bone meal, feather meal and other types of meal produced from other slaughterhouse waste, for example poultry chicken meal. These are typically cheaper than fish meal and fish oil. However, in some geographic regions, such as Europe, there has been a restriction against using such raw materials in the production of feeds for food-producing animals and fish. Insect meal and protein from microbial and microalgal biomass are known for this purpose also, and macroalgae may be used in future.

It is also known to use vegetable protein such as wheat gluten, maize (corn) gluten, soybean based products, lupin meal, pea meal, bean meal, rape meal, sunflower meal, distiller grains solubles (DDGS), faba bean products and rice flour. Soya is a low price raw material with high protein content and is available in very large quantities on a world-wide basis. Therefore, soya has been used in fish feeds for many years.

There is thus pressure to minimise the quantity of raw material used in fish feed for aquaculture.

In addition, aquaculture is capital intensive. There are investments in cages, pens or ponds, feeding automata, storage facilities and other infrastructure. The fish themselves have associated costs as they are purchased as fingerlings (e.g. trout and salmon species) or wild caught.

The most important single cost in aquaculture is the cost of the feed. Labour costs are also important.

The selling price of the fish and the number of fish that are harvested determine the profitability of the operation.

A faster turnover has several positive results. First, it helps cash flow. Second, it improves risk management. Fish diseases are common, and the likelihood of an outbreak is higher over a long growing period. There is also a risk that fish will escape due to accidents, e.g. when shifting nets, or due to bad weather causing wrecked fish pens.

Turnover rate is determined by how fast the fish grow to a harvestable size. As an example, it takes from 12 to 18 or even 24 months to raise Atlantic salmon from smolt (seawater transfer stage, discussed in more detail below) to harvestable size. Harvestable size is dependent on the fish species and market. Some markets for Atlantic salmon prefer fish larger than 6 kg. Rainbow trout is in some markets sold as portion sized and the weight is 300 g. Farming of larger rainbow trout also takes place.

Growth rate is expressed as percentage increase in body mass from day to day (Specific Growth Rate, SGR). This is calculated as:

$$SGR = ( {( \frac{FinalW}{InitW} )}^{1 / Days} - 1 ) \cdot 100$$

FinalW=final weight
InitW=initial weight
Days=time from measuring initial weight to final weight

The SGR does not take into account the amount of feed fed to obtain growth. It is a measure of growth rate only. A high SGR is dependent on the digestibility of the raw materials and how optimal the feed composition is with respect to protein and fat ratio, amino acid composition and composition of fatty acids. Microingredients such as vitamins and minerals must also be present in sufficient quantities.

Thus, it is desirable to produce a fish feed which leads to good (high) SGR. High feed intake, body weight and weight gain are all also desirable.

Particular feed considerations apply to salmonids, which are anadromous fish. Anadromous fish hatch in freshwater and spend the fry phase in freshwater, but after smoltification (i.e. reaching the physiological stage of smolt, when they can first be transferred from freshwater to seawater) migrate to brackish water and possibly seawater having full salinity. The fish return to freshwater for spawning. By salmonids is meant species belonging to the family Salmonidae. Examples of salmonids are salmon species such as Atlantic salmon (Salmo salar), and trout species such as rainbow trout.

The smolt transformation of anadromous fish involves changes in behaviour, morphology and physiology that are preparatory for, and will improve success in, migration and seawater entry.

Smoltification in aquaculture can be achieved by photomanipulation i.e. lighting regimes. The traditional method is “winter signal” i.e. a part of the day light and a part of the day darkness, for example about 12 hours darkness per day and about 12 hours light per day. However, winter signal (i.e., keeping fish in the dark for a period of time during the day) reduces feed intake and therefore growth compared with other lighting regimes, particular 24:0, i.e. continuous light. It is desirable to develop other feeds and feeding methods that support feed intake and/or growth in the freshwater phase, for example using specific feeds.

WO02/30192 of Aquabio Products Sciences LLC (relating to a feed product known as “SUPERSMOLT”) discloses a smoltification method requiring both a feed containing sodium salt and polyvalent cation receptor modulator (PVCR), and addition of Ca²⁺ and Mg²⁺ ions to the water. The preferred PVCR is tryptophan in free amino acid form. A feed containing histidine in free amino acid form as PVCR was tested but reported to give poor results (Table 19). This 20 document reviews the physiology of smoltification.

WO2016/046182 of Europharma A/S (relating to a feed product known as “SUPERSMOLT FeedOnly”, developing the earlier SUPERSMOLT work) discloses a fish feed useful in a method for smoltification and prevention of desmoltification in Salmonidae, comprising sodium salts, magnesium salts and calcium salts, and also a polyvalent cation receptor modulator (PVCR) which may be tryptophan in free amino acid form.

In commercial farming of salmon and rainbow trout it is well known that the fish lose their appetite when they as smolt are ready for seawater and are transferred from freshwater to seawater. This is a stressful period associated with high mortality. The fish may mope for several weeks after the transfer. For the fish farmer this means lost growth. It takes longer to get the fish to a size ready for butchering, particularly because growth has a daily compound effect. In fish farming there is therefore a need for a feed which is readily accepted by fish recently transferred to seawater.

WO2010087715 of the applicant group of companies discloses a fish feed where the fish feed is produced by extrusion and contains at least 3 percent by weight of arginine. Some of the arginine may be provided in free amino acid form e.g. by supplementation with 1 wt % crystalline arginine. The feed is used to prevent reduced growth of salmonids at transfer from freshwater to seawater. This document contains a review of the literature on arginine requirements of salmon.

Also, salmonids are liable to develop cataracts (opacification of the lens and/or lens capsule) in their eyes. This leads to reduced vision which can cause reduced feed intake. EP1199947 of BioMar Group discloses a salmonid feed for reduction of cataracts including at least 1.15 wt % histidine. It is desirable to develop feeds and feeding methods, especially for the freshwater phase, that reduce cataract and/or support feed intake and/or growth in the saltwater phase, for example using specific feeds.

### SUMMARY

In a first aspect, the invention relates to a fish feed for feeding to anadromous fish in freshwater comprising arginine in free amino acid form or dipeptide form, preferably in free amino acid form, histidine in free amino acid form or dipeptide form, preferably in free amino acid form, and 0.2-5 wt % Na⁺.

Preferably, the fish feed comprises 0.3-4.5 wt %, more preferably 0.4-4 wt %, yet more preferably 0.6-3.5 wt %, such as 0.8-3 wt %, or 1-2.5 wt %, Na⁺.

Preferably, at least part of said Na⁺ is added to the feed in the form of NaCl, more preferably in the form of 1-10 wt %, or preferably 3-8 wt %, NaCl.

The term “fish feed” as used herein includes compositions as described below. Typically, fish feed includes fish meal as a component. Suitably, fish feed is in the form of flakes or pellets, for example extruded pellets. Preferred pellet sizes (diameter) are in the range of 1.5-6 mm e.g. 2-5.5 mm, 2.5-5 mm, 3-4.5 or 3.5-4 mm.

The references to the amino acids arginine and histidine each include D and L isomers, racemic or non-racemic mixtures, and salts thereof. Preferably, L-arginine and L-histidine or dipeptides or salts thereof are used.

Preferably, the stated amounts of arginine and histidine in free amino acid form or dipeptide form are added amounts, in addition to any contribution from raw ingredients such as fish meal.

Preferably, each of the arginine and histidine in free amino acid form or dipeptide form is synthetic and/or is provided in a form which is more than 50% pure. Suitably, histidine may be provided in the form of histidine hydrochloride monohydrate.

Preferably, the fish feed comprises 0.5-7 wt %, preferably 0.6-5 wt %, more preferably 0.7-4 wt %, yet more preferably 0.75-3 wt %, even more preferably 0.8-2 wt %, such as 1-1.5 wt %, arginine in free amino acid form or dipeptide form, preferably free amino acid form, and/or 0.1-7 wt %, preferably 0.15-5 wt %, more preferably 0.2-4 wt %, yet more preferably 0.25-3 wt %, such as 0.3-2 wt %, or 0.35-1.5 wt %, histidine in free amino acid form or dipeptide form, preferably free amino acid form.

Typically, fish feed, particular fish feed for administration in the freshwater phase, comprises fish meal. Fish meal in turn comprises Ca²⁺ ions and Mg²⁺ ions. Tacon and Da Silva (Mineral composition of some commercial fish feeds available in Europe. Aquaculture. 1983, vol. 31:11-20) provide an analysis of mineral composition of 38 commercially available fish feeds. It was found that such feeds comprise Ca²⁺ ions in the range of 2.5-37 g/kg, and Mg²⁺ ions in the range of 0.4-3.0 g/kg, preferably 1-2.5 g/kg. Suitably, the fish feed of the invention may comprise such conventional levels of Ca²⁺ ions and Mg²⁺ ions.

In an embodiment, a maximum of 1 wt % Ca²⁺ salt and/or Mg²⁺ salt is added to the feed on top of conventional levels and preferably no Ca²⁺ salt or Mg²⁺ salt is added to the feed on top of conventional levels.

In a preferred embodiment, the fish feed comprises 0.75-1.5 wt % arginine in free amino acid form or dipeptide form, preferably in free amino acid form, 0.25-0.75 wt % histidine in free amino acid form or dipeptide form, preferably in free amino acid form, and 1-3 wt % Na⁺.

Preferably, the anadromous fish are salmonids, more preferably salmon e.g. Atlantic salmon.

Salmonids are so-called oily fish. They require high lipid feed to remain healthy. They deposit fat in the fillet. Generally they can make use of a large share of the fat in the feed to energy, while the protein in the feed is deposited in the musculature. This means that a high share of the supplied protein is utilised for growth. This is favourable because it gives an advantageous ratio between used feed and saleable product. Preferably, therefore the fish feed contains at least 15 wt % lipid, more preferably at least 20 wt % lipid, for example, 20 to 40 wt %, 20 to 35 wt %, or 20 to 30 wt % lipid. A preferred range is 20 to 35 wt % lipid.

Preferably, the fish feed contains a protein level of 30 to 60 wt %, preferably 35 to 55 wt %, more preferably 36 to 54 wt %, even more preferably 37 to 53 wt %, yet more preferably 38 to 52 wt %, or 39 to 52 wt %, such as 40 to 52 wt %.

In a preferred embodiment, the fish feed has a proximate composition of 30-50 wt % protein, 3-15 wt % moisture and lipid as described above.

When comparing the nutritional content of different feeds and type of feed it is important to take the water content into consideration. For pressed feed and extruded feed it is normal in commercial context, and also in many scientific papers, to state the feed composition on an “as is” basis, and this approach is used here. For feed containing a lot of and/or varying amounts of water it is normal to state the composition based on the dry substance.

Preferably, the fish feed comprises one or more of:
sources of protein, carbohydrate and lipid as discussed in more detail below;
Optional binder (for example starch; suitable sources are wheat, potato flour, tapioca flour, faba beans, pea starch, barley and corn starch)
vitamin premix;
mineral premix; and
pigment (for example canthaxanthin, astaxanthin; sources of mixed pigments can be used);
optional further functional ingredients e.g. immune stimulants, palatability enhancers, faecal binders.

Suitable sources of protein, carbohydrate and lipid include:
fish meal and fish oil
krill meal and krill oil
microalgae and macroalgae
animal meal (for example blood meal, feather meal, poultry meal e.g. chicken meal, bone meal, insect meal and/or other types of meal produced from other slaughterhouse waste)
animal fat (for example poultry oil)
vegetable meal (for example soya meal, lupin meal, pea meal, bean meal, rape meal, rice meal, linseed meal, sunflower meal)
vegetable oil (for example rapeseed oil, soya oil, linseed oil, sunflower oil)
gluten (for example wheat gluten or corn gluten)
further added amino acids (for example lysine, methionine))

In addition, some suitable sources of protein, carbohydrate and lipid are discussed in the introduction.

The fish feed is preferably made by a method comprising the steps of:
mixing ingredients in a mixer;
extrusion or pressing of pellets; and
coating the pellets with oil.

Preferably, the fish feed is extruded.

Extruders of the single screw and double screw types are suitable. Suitably a cooking extrusion process is used, which is typically as follows. The material extruded is a mixture of ingredients as described above, and water. The water may be added to the mixture in the form of water or steam. The mixture may be heated beforehand in a so-called preconditioner where the heating takes place by adding steam to the mixture. Steam and water may also be added to the mass inside the extruder. In the extruder itself the pasty mass is forced by means of the screws toward a constriction in the outlet end of the extruder and further through a die plate to form a desired cross-sectional shape. On the outside of the die plate a rotating knife is normally positioned cutting the string coming out of the die holes to a desired length. Normally the pressure on the outside of the die plate will be equal to the ambient pressure. The extruded product is referred to as extrudate. Due to the pressure created inside the extruder, and the addition of steam to the mass, the temperature can exceed 100° C. and the pressure will be above atmospheric pressure in the mass before it is forced out of the die openings.

Cooking extrusion of material containing starch causes the starch granules to swell so that the crystalline starch in the granules is released and may unfold. This is referred to as gelatinisation of the starch. The starch molecules will form a network contributing to hold the extrudate together. Particularly in feed for carnivorous fish, starch-containing raw materials are added due to their properties as binding agent in the finished fish feed. The natural prey for carnivorous fish does not contain starch. Carnivorous fish have little or no digestive enzymes that may alter the starch to digestive sugars. Cooking of the starch makes it more digestible. This is partly due to the starch no longer being raw, and partly because the cooking process starts a decomposition of the starch to smaller sugar units being easier to digest.

A further effect of the cooking extrusion is that the extrudate becomes porous. This is caused by the pressure drop and temperature drop over the die opening. The water in the extrudate will immediately expand and be freed as steam and leave behind a porous structure in the extrudate. This porous structure may be filled with oil in a later process stage. An extruded feed will typically contain between 18 and 30% of water after extrusion. After extrusion this feed undergoes a drying stage and a subsequent stage of oil coating. The end product typically contains approx. 10% of water or less and will thus be storage stable as the water content in such feed is so low that growth of fungus and mould is prevented and also bacterial decay is avoided. After oil coating the feed is typically cooled and packaged.

The extrudate is thus different from a pressed feed. By a pressed feed is meant feed produced by means of a feed press. This process differs from extrusion in many ways, typically as follows. Less water and steam is utilised in the process. The feed mixture is forced through a die ring from the inside out by means of rollers rotating on the inside of the die ring. The temperature and pressure are lower than at extrusion and the product is not porous. The process has the effect that the starch is not as digestible as after extrusion. A pressed feed will normally contain less than 15% water after pressing and possible oil application. It is not usually necessary to dry a pressed feed, but post-conditioning may be applied. The feed is cooled before packaging.

It is common to feed with only one type of feed, in which case each piece of feed should be nutritionally adequate. However, two or more types of feed may be used in combination.

In a second aspect, the invention relates to a method of feeding anadromous fish in freshwater, comprising feeding the fish with a fish feed as described above for a feeding period.

The term “freshwater” as used herein includes water with less than 3000 ppm total dissolved salts, e.g. less than 0.05 ppt NaCl. The term “freshwater” as used herein includes water with less than 1 ppt total dissolved salts.

As in the first aspect of the invention, the fish are preferably salmonids, more preferably salmon, most preferably Atlantic salmon.

Suitably, the feeding period is at least 250 day degrees, preferably at least 350 day degrees, for example at least 450 day degrees, or at least 550 day degrees, or at least 650 day degrees, more preferably at least 750 day degrees, or at least 850 day degrees, at least 950 day degrees, or at least 1000 day degrees. Preferably, the feeding period is in the range of 420-1008 day degrees.

The term “day degrees” (also referred to as “degree days”) as used herein refers to the number of days multiplied by the average water temperature in ° C. The term “seawater” as used herein includes water of high salinity e.g. brackish water. Seawater may comprise brackish water e.g. from 3 ppt to 30 ppt of dissolved salts, and seawater may comprise full strength seawater from 30 ppt to 38 ppt of dissolved salts.

Suitably, the feeding period is at least 5 weeks, preferably at least 12 weeks. The feeding period preferably ends within one week before transfer of the fish to seawater but can end earlier e.g. 5 weeks before transfer. Alternatively, the feeding period may be up to transfer of the fish to seawater. For an average water temperature of 12° C., 420 day degrees corresponds to 5 weeks and 1008 day degrees corresponds to 12 weeks.

An artificial winter signal may be applied during at least a part of the feeding period and/or a continuous light signal may be applied during at least a part of the feeding period.

The term “winter signal” as used herein refers to any use of light/darkness, other than full 24 hour light. Non-limiting examples of winter light signals are 6-12 hours of light and 18-12 hours darkness per day, or 12-18 hours light and 6-12 hours darkness per day. A 12:12 winter signal is commonly used and considered suitable. The light may be artificial light (artificial winter signal) or natural light (natural winter signal).

In one preferred embodiment, initially an artificial winter signal is applied and then a continuous light signal is applied. Preferably, the artificial winter signal is applied for 5-9 weeks e.g. 7 weeks, followed by a continuous light signal for 3-7 weeks e.g. 5 weeks.

In another preferred embodiment, a continuous light signal is applied throughout the feeding period.

Further aspects of the invention include:
a method of increasing seawater tolerance of anadromous fish comprising feeding the fish according to the method described above;
a method of promoting growth of anadromous fish in the freshwater and/or seawater phase, comprising feeding the fish according to the method described above;
a method of promoting feed intake of anadromous fish in the freshwater and/or seawater phase, comprising feeding the fish according to the method described above;
a fish feed as described above for use in preventing or reducing severity of cataracts in fish, the fish feed optionally being fed to the fish according to the method described above;
a method of preparing a fish feed as described above, the method comprising mixing ingredients, and optionally extruding pellets of the fish feed, wherein the free amino acids are mixed into the ingredients and/or added by top coating.

“Promoting growth” as used herein refers to achieving better growth compared with fish not receiving the test diet.

Features described in relation to any aspect of the invention may be used in any other aspect of the invention.

### BRIEF DESCRIPTION OF THE DRAWINGS

The invention will be further described with reference to the non-limiting example and drawings, in which:

FIG. 1 shows water and lighting regimes for the example.

FIG. 2 shows feed, water and lighting regimes for the example.

FIG. 3 shows feed intake results (as percentage of body weight per day, and as accumulated feed intake) for the example.

FIG. 4 shows body weight results for the example.

FIG. 5 shows weight gain results for the example.

FIG. 6 shows specific growth rate (SGR) results for the example.

FIG. 7 shows cataract results for the example.

### DETAILED DESCRIPTION OF THE DRAWINGS

### Example

A trial was conducted at Skretting ARC Lerang Research Station, Norway, between November 2020 and May 2021.

Atlantic salmon having an average starting weight of 40 g were distributed into experimental 100 L tanks, starting with 80 fish per tank and 3 tanks per diet (24 tanks total).

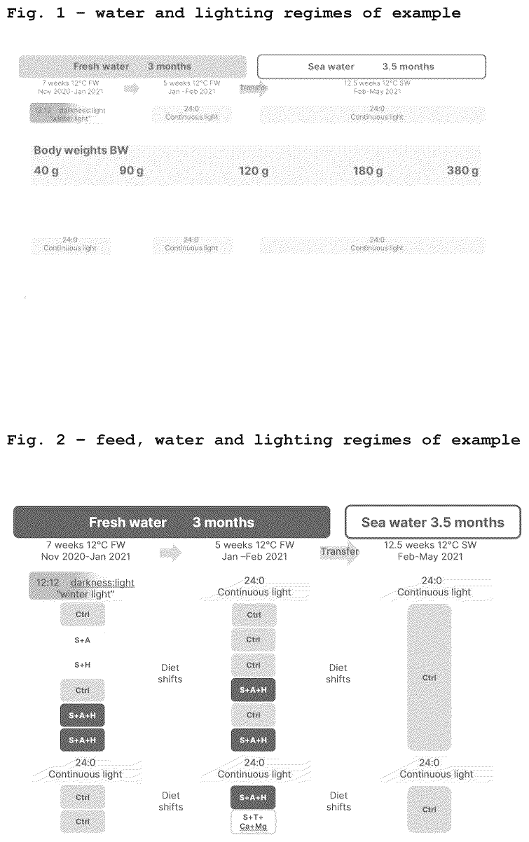

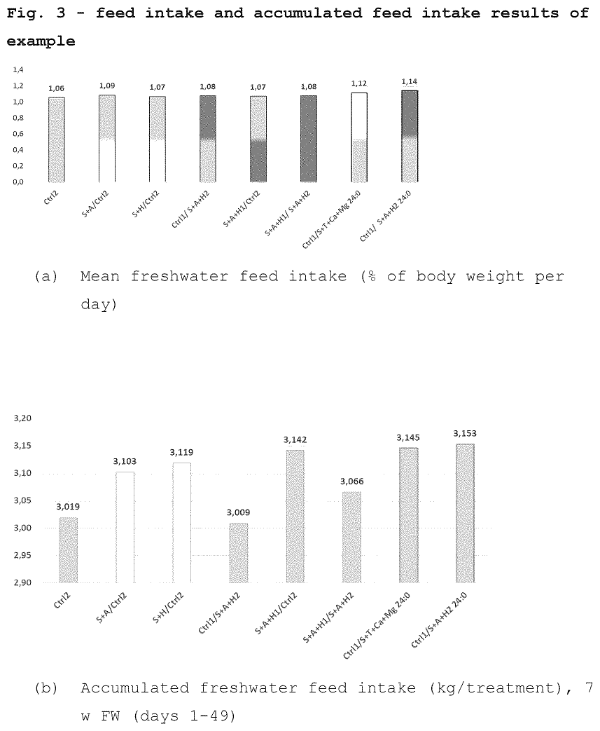

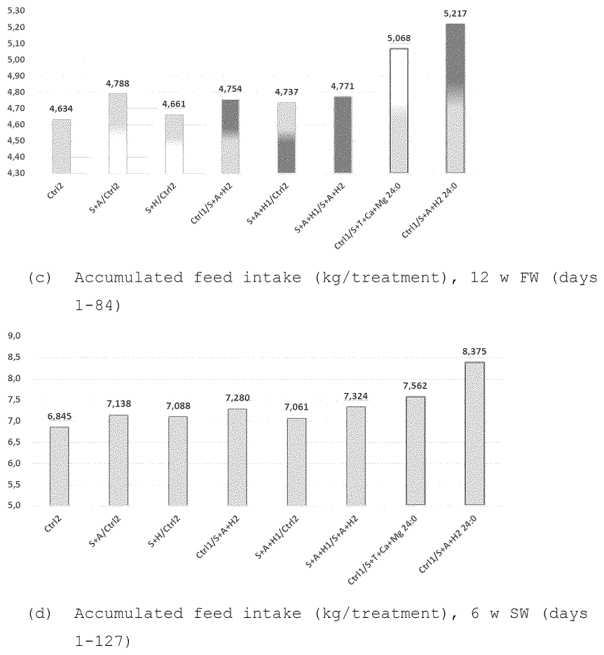

The feed types, water type and temperature, feeding times and lighting regimes were as shown in Tables 1 and 2 and FIGS. 1 and 2. Overall there was a 3 month period in freshwater followed by seawater transfer and a 3.5 month period in seawater. Different lighting regimes were used for the first 7 weeks in freshwater (12:12 darkness: light i.e. winter signal, or 24:0 light i.e. continuous light). Thereafter all groups were in continuous light.

Feeding after seawater transfer (when daily feed intake could not be measured) aimed at 1.15% of the body weight per day. Before seawater transfer daily feed intake was measured and is reported below.

The average final weight after 3.5 months in seawater was 381 g.

### Feeds

The feeds were produced by extrusion. Arginine and histidine were added into the dry premix.

The feeds used in the example are shown in Table 1:

TABLE 1

| | [symbol] | | | | | | | |
|---|---|---|---|---|---|---|---|---|
| | [symbol] , g | | | | | | | |
| | Ctr 1 | S + A | S + H | S + A + H | Ctrl2 | S + T + Ca + Mg | S + A + H2 | Ctrl SW diet |
| | [symbol] | [symbol] | [symbol] | [symbol] | [symbol] | [symbol] | [symbol] | [symbol] |
| | [symbol] size, mm | | | | | | | |
| | [symbol] | | | | | | | |
| | 2 | 2 | 2 | 2 | 2 | 2 | 2 | 3 |
| | FW | FW | FW | FW | FW | FW | FW | FW |
| [symbol] | 5.24 | [symbol] | 1 [symbol] .5 [symbol] | 1 [symbol] . [symbol] | 7.00 | 1 [symbol] .5 [symbol] | 16.5 [symbol] | 7 [symbol] |
| [symbol] | | | | | | | | |
| [symbol] | 25.00 | [symbol] .7 [symbol] | 11.5 [symbol] | 5.94 | 22.22 | 1 [symbol] .83 | [symbol] 1.79 | 21. [symbol] 5 |
| [symbol] | [symbol] | [symbol] | [symbol] 1 | [symbol] 1 | [symbol] 1 | [symbol] 1 | [symbol] 1 | [symbol] 1 |
| [symbol] | [symbol] .26 | [symbol] .5 [symbol] | [symbol] .48 | 9.84 | [symbol] .34 | [symbol] .96 | [symbol] 0.05 | [symbol] .37 |
| [symbol] | 35.00 | 35.00 | 35.00 | 35.00 | 35.00 | 35. [symbol] 0 | 35.0 [symbol] | 35. [symbol] |
| Vegetable oil | [symbol] .26 | [symbol] .5 [symbol] | [symbol] .48 | 9.84 | [symbol] .34 | [symbol] .96 | [symbol] 0.05 | [symbol] .37 |
| [symbol] | 0.85 | 1.0 [symbol] | [symbol] 1 | 1.05 | 1.00 | 1.13 | 1.14 | 1.00 |
| Salt NaCl | [symbol] .00 | 5.0 [symbol] | 5.00 | 5.00 | [symbol] . [symbol] 0 | 5. [symbol] 0 | 5.0 [symbol] | [symbol] . [symbol] 0 |
| [symbol] | [symbol] | [symbol] | [symbol] .5 [symbol] | [symbol] .5 [symbol] | [symbol] | [symbol] | [symbol] .5 [symbol] | [symbol] |
| Water, [symbol] | [symbol] 45 | 1.26 | 1.12 | 1.32 | [symbol] .44 | 1.39 | 1.46 | [symbol] .42 |
| A [symbol] [symbol] % | [symbol] | 1.25 | [symbol] | 1.25 | [symbol] | [symbol] | 1.25 | [symbol] |
| [symbol] | 14.53 | 9.65 | 10.3 [symbol] | [symbol] .84 | 15.66 | [symbol] .01 | 7.27 | 16.29 |
| [symbol] % | [symbol] | [symbol] | [symbol] | [symbol] | [symbol] | [symbol] .4 [symbol] | [symbol] | [symbol] |
| [symbol] % | [symbol] | [symbol] | [symbol] | [symbol] | [symbol] | 0.25 | [symbol] | [symbol] |
| [symbol] % | [symbol] | [symbol] | [symbol] | [symbol] | [symbol] | 0.75 | [symbol] | [symbol] |
| Total | 100.0 | 100.0 | 100.0 | 100.0 | 100.0 | 100.0 | 100.0 | 100.0 |

* L-histidine monohydrochloride monohydrate
Base recipes corresponded to the relevant control recipe, with minimum necessary modifications.
Diet S + T + Ca + Mg was designed to resemble the “SUPERSMOLT FeedOnly” feed discussed above. [symbol]
indicates data missing or illegible when filed

### Stage 1: Freshwater, 7 Weeks, Target Weight Range 40-90 g, 40 g Initial Mean Fish Weight, 92 g Final Mean Fish Weight, 2 mm Diets

$$Ctrl 1 - Control$$

$$Feed A - base recipe + 1.25 wt \% Arg + 5 wt \% NaCl$$

$$Feed S + H - base recipe + 0.5 wt \% His . HCl monohydrate ( 0.37 wt \% His ) + 5 wt \% NaCl$$

$$Feed S + A + H 1 - base recipe + 1.25 wt \% Arg + 0.5 wt \% His . HCl monohydrate ( 0.37 wt \% His ) + 5 wt \% NaCl ( feed of the invention )$$

### Stage 2: Freshwater, 5 Weeks, Target Weight Range 90-120 g, 92 g Initial Mean Fish Weight, 121 g Final Mean Fish Weight, 2 mm Diets

$$Ctrl 2 - control$$

$$Feed S + A + H 2 - base recipe + 1.25 wt \% Arg + 0.5 wt \% His . HCl monohydrate ( 0.37 wt \% His ) + 5 wt \% NaCl ( feed of the invention )$$

$$Feed S + T + Ca + Mg - resembling “ Supersmolt FeedOnly ” including 0.4 wt \% L - Trp + 0.25 wt \% {MgCl}_{2} + 0.75 wt \% {CaCl}_{2} + 5 wt \% NaCl ( comparative example )$$

### Stage 3: Seawater, 3.5 Months

Ctrl SW diet according to table 2 for 3.5 months in seawater.

Proximal analysis for the feeds is shown in Table 2:

TABLE 2

| | | Ctrl1 | S + A | S + H | S + A + H | Ctrl2 | S + T + Ca + Mg | S + A + H2 | CtrlSW |
|---|---|---|---|---|---|---|---|---|---|
| Moisture | % | 8.0 | 8.0 | 8.0 | 8.0 | 8.0 | 8.0 | 8.0 | 8.0 |
| Crude Protein | % | 46.0 | 46.6 | 45.8 | 47.0 | 45.7 | 45.5 | 46.8 | 45.3 |
| Crude fat | % | 22.6 | 23.4 | 23.3 | 23.6 | 22.8 | 24.2 | 24.4 | 22.8 |
| Ash | % | 7.2 | 11.6 | 12.1 | 12.0 | 7.2 | 12.7 | 12.2 | 7.2 |
| Total Arginine | g/kg | 27.0 | 35.2 | 23.4 | 35.2 | 26.2 | 23.3 | 35.6 | 25.9 |
| Total Histidine | g/kg | 11.7 | 11.0 | 14.7 | 14.6 | 11.6 | 11.1 | 14.7 | 11.5 |

For all treatment regimes, mortality in both the freshwater phase and the seawater phase was very low with no mortality in the first phase in freshwater (Stage 1), 1-2 fish per tank in the last phase of freshwater (Stage 2), no mortality in the first 6 weeks in seawater and one dead fish in one tank during week 7-12 in seawater (Stage 3). No statistically significant differences were observed between any of the treatment regimes.

Daily feed intake results are shown in FIG. 3. Regime Ctrl1/S+A+H2/24:0 gave the best feed intake results in freshwater (1.14% of body weight per day), in seawater (0.91 wt % of body weight) and overall for freshwater and seawater (1.06% of body weight). These results were better than Ctrl1/S+T+Ca+Mg/24:0 (1.12%, 0.76% and 1.00% of body weight per day in freshwater, seawater and overall respectively). The best regime for feed intake with initial 12:12 lighting was S+A+H1/S+A+H2 (1.01% of body weight per day overall). Ctrl1/S+A+H2 also performed well.

Accumulated feed intake results are also shown in FIG. 3. Regime Ctrl1/S+A+H2/24:0 gave the best accumulated feed intake results in freshwater (3.153 kg for Stage 1; 5.217 kg for freshwater overall) and in seawater (8.375 kg for first 6 weeks). These results were better than Ctrl1/S+T+Ca+Mg/24:0 (3.145 kg, 5.068 kg and 7.562 kg respectively). The difference in seawater accumulated feed intake was particularly large. Of the regimes with initial 12:12 lighting, S+A+H1/S+A+H2 gave good accumulated feed intake results in seawater (7.324 kg).

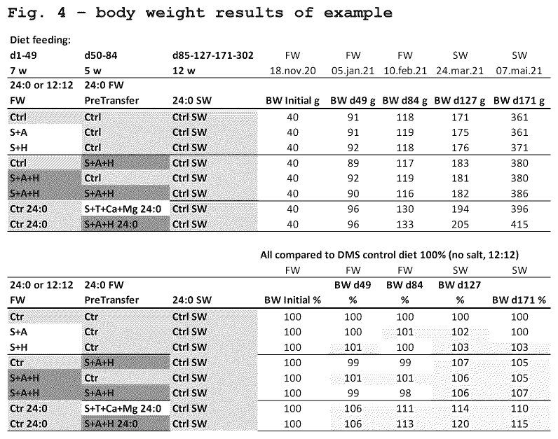

Body weight results are shown in FIG. 4. Regime Ctrl1/S+A+H2/24:0 gave the best body weight results from day 84 onwards. These results were better than Ctrl1/S+T+Ca+Mg/24:0. The best regime for body weight at final day after 3.5 months in seawater with initial 12:12 lighting was S+A+H1/Ctrl2. Regimes with initial 12:12 lighting generally perform as well as or better than Ctrl1/S+T+Ca+Mg with 24:0 lighting throughout by day 171.

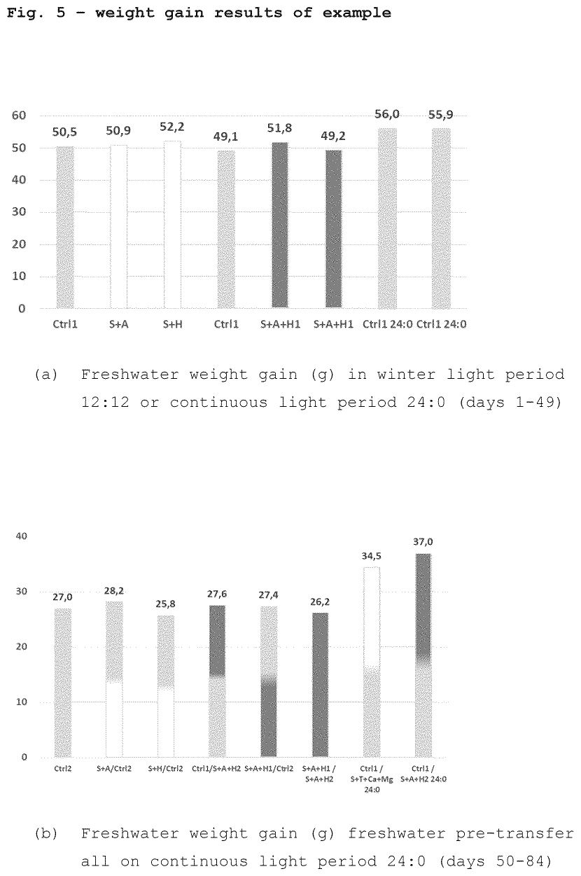

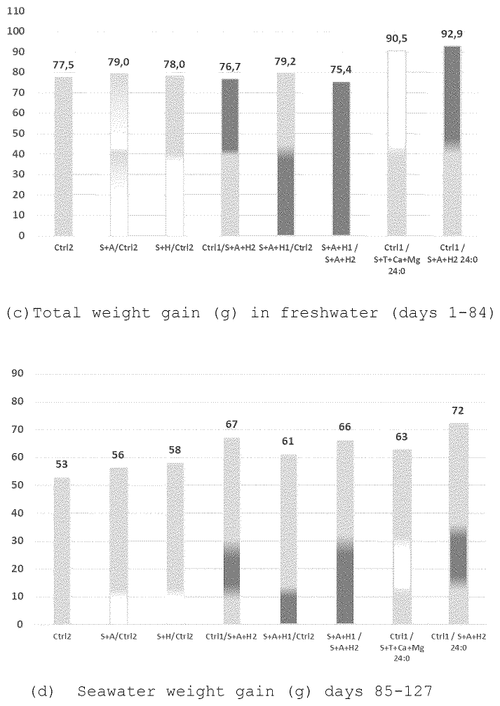

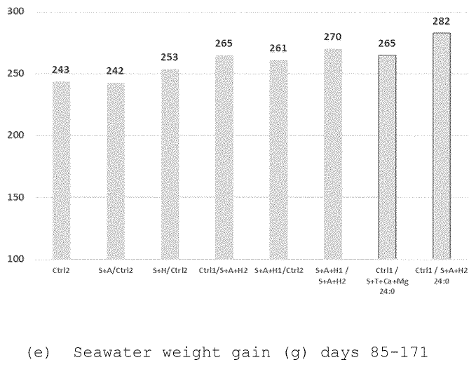

Weight gain results are shown in FIG. 5. Again, Ctrl1/S+A+H2/24:0 gave the best growth results in freshwater (total weight gain in freshwater 92.9 g) and seawater (total weight gain in seawater 282 g). These results were better than with the corresponding regime with Ctrl1/S+T+Ca+Mg/24:0 (90.5 g and 265 g respectively). The best regime with initial 12:12 lighting was S+A+H1/Ctrl2: this regime gave a total weight gain in freshwater of 79.2 g and a total weight gain in seawater of 261 g. During the first 6 weeks post-transfer to seawater (days 85-127), regime Ctrl1/S+A+H2 gave a 26% higher weight gain than the control (67 g compared with 53 g). This is important because, as explained above, growth is often poor in this critical post-transfer period.

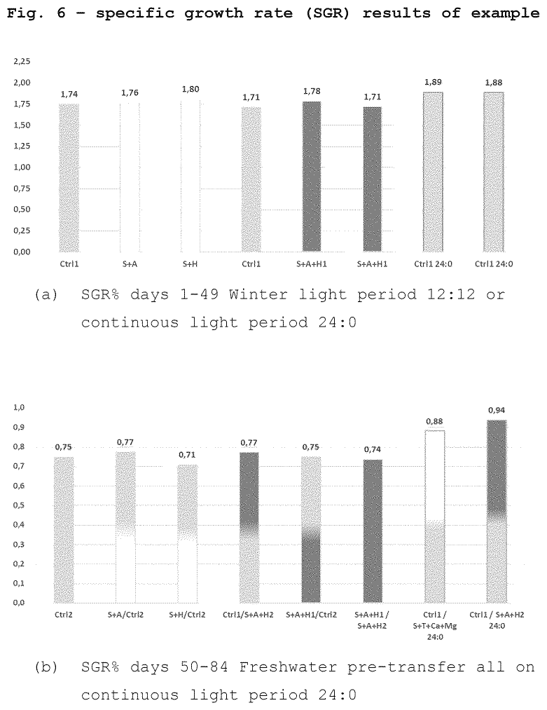

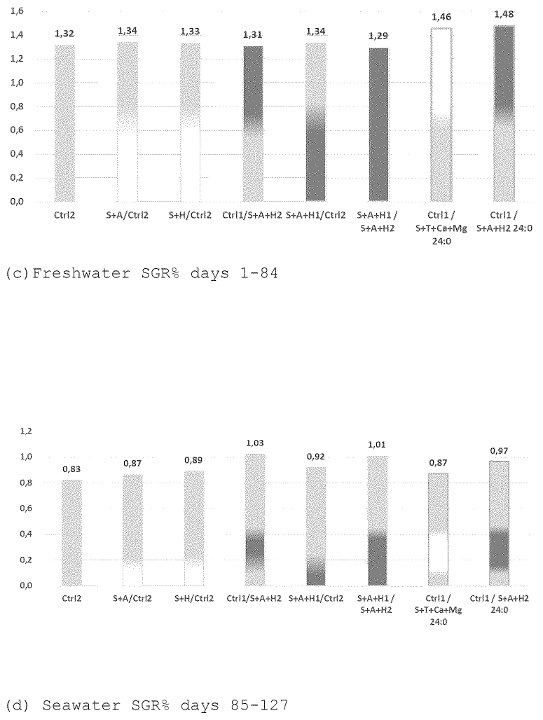

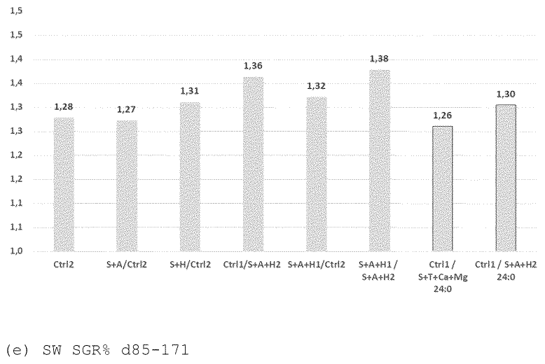

Specific growth rate (SGR) results are shown in FIG. 6. Again, Ctrl1/S+A+H2/24:0 gave the best SGR results in freshwater (1.48%) and better SGR in seawater for the first 6 weeks post-transfer (days 85-127) than Ctrl1/S+T+Ca+Mg/24:0 (0.97% compared with 0.87%). Regimes with initial 12:12 lighting and feeds of the invention (Ctrl1/S+A+H2; S+A+H1/Ctrl2; S+A+H1/S+A+H2) performed well for seawater SGR. During the first 6 weeks post-transfer to seawater (days 85-127), Ctrl1/S+A+H2 gave a 24% higher SGR than the control (1.03% compared with 0.83%). This is important because, as explained above, growth is often poor in this critical post-transfer period.

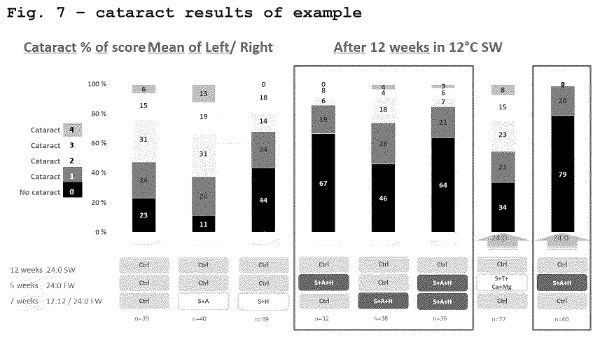

Cataract results are shown in FIG. 7. Again, Ctrl1/S+A+H2/24:0 gave the best cataract results (cataract score 0 for 79% of fish). These results were better than with the corresponding regime with Ctrl1/S+T+Ca+Mg/24:0 (cataract score 0 for 34% of fish). The best regime with initial 12:12 lighting was Ctrl1/S+A+H2: this regime gave a cataract score 0 for 67% of fish. This compares favourably with the regime using His-containing feed i.e. S+H/Ctrl2 (cataract score 0 for 44% of fish).

Enzymatic Nak ATPase results were assessed. Freshwater gill samples were analysed by Sintef Norlab, Norway. All three regimes with S+A+H2 in Stage 2 (freshwater) gave high enzymatic activity results at day 84, immediately before seawater transfer. This demonstrated that the fish were ready to be transferred to seawater. These results were better than with the corresponding regime with Ctrl1/S+T+Ca+Mg/24:0. Good results were achieved with both lighting regimes; all fish were ready to be transferred to seawater.

The example shows that the freshwater Atlantic salmon feeds of preferred embodiments of the invention led to good and efficient growth in both freshwater and seawater (as measured by feed intake, body weight, weight gain and specific growth rate), timely smoltification/good readiness for transfer to seawater and avoidance of cataracts. Growth was particularly good in the critical 6 week period after seawater transfer. Good results were achieved with both 12:12 and 24:0 initial lighting regimes. Feeding with a feed of the invention in the period immediately before seawater transfer gave good results. The results were comparable with or better than regimes with Ctrl1/S+T+Ca+Mg/24:0.

Whilst the invention has been described with reference to preferred embodiments, it will be appreciated that various modifications are possible within the scope of the invention.

1. A fish feed for feeding to anadromous fish in freshwater comprising arginine in free amino acid form or dipeptide form, 0.1 to 7 wt % histidine in free amino acid form or dipeptide form, and 0.25 to 5 wt % Na+.

2. The fish feed as claimed in claim 1, comprising 0.5 to 7 wt %, arginine in free amino acid form or dipeptide form, and/or 0.2 to 1.5 wt %, histidine in free amino acid form or dipeptide form.

3. The fish feed as claimed in claim 1, wherein a maximum of 1 wt % Ca2+ salt and/or Mg2+ salt is added to the feed.

4. The fish feed as claimed in claim 1, comprising 0.6 to 1.25 wt % arginine in free amino acid form, 0.2 to 0.75 wt % histidine in free amino acid form, and 1 to 3 wt % Na+.

5. The fish feed as claimed in claim 1, comprising at least 15 wt % lipid.

6. The fish feed as claimed in claim 1, wherein the fish feed is in the form of extruded pellets.

7. A method of feeding anadromous fish in freshwater, comprising feeding the fish with a fish feed comprising arginine in free amino acid form or dipeptide form, histidine in free amino acid form or dipeptide form, and 0.25 to 5 wt % Na+ for a feeding period.

8. The A method as claimed in claim 7, wherein the fish are salmonids.

9. The method as claimed in claim 7, wherein the feeding period is at least 250 day degrees.

10. The method as claimed in claim 7, wherein an artificial and/or natural winter signal is applied during at least a part of the feeding period and/or a continuous light signal is applied during at least a part of the feeding period.

11. The method as claimed in claim 10, wherein initially an artificial winter signal is applied and then a continuous light signal is applied.

12. The method as claimed in claim 10, wherein a continuous light signal is applied throughout the feeding period.

13. A method of increasing seawater tolerance of anadromous fish comprising feeding the fish according to the method of claim 7.

14. A method of promoting growth of anadromous fish in the freshwater and/or seawater phase, comprising feeding the fish according to the method of claim 7.

15. A method of promoting feed intake of anadromous fish in the freshwater and/or seawater phase, comprising feeding the fish according to the method of claim 7.

16. A fish feed comprising arginine in free amino acid form or dipeptide form, histidine in free amino acid form or dipeptide form, and 0.25 to 5 wt % Na+ for use in preventing or reducing severity of cataracts in fish.

17. A method of preparing a fish feed as claimed in claim 1, the method comprising mixing ingredients, and optionally extruding pellets of the fish feed, wherein the free amino acids are mixed into the ingredients and/or added by top coating.

18. A method as claimed in claim 17 of preparing a fish feed, wherein the arginine and histidine in free amino acid form or dipeptide form are synthetic and/or are provided in a form which is more than 50% pure.

19. The fish feed as claimed in claim 2, wherein a maximum of 1 wt % Ca2+ salt and/or Mg2+ salt is added to the feed.

20. The method as claimed in claim 8, wherein the feeding period is at least 250 day degrees.
